(19) United States
(12) Patent Application Publication
(10) Pub. No.: US 2013/0046128 A1
(43) Pub. Date: Feb. 21, 2013

(54) Radiotherapy Delivery Cannula With Visual Confirmation Window

(71) Applicant: NeoVista, Inc., Newark, CA (US)

(72) Inventors: David Gyorke, El Cerrito, CA (US); Benjamin Woodward, Santa Clara, CA (US); John N. Hendrick, Henderson, NV (US); John P. Dunbar, Tijeras, NM (US); Michael D. Tocci, Sandia Park, NM (US); Jay Daulton, GIlroy, CA (US); William Metzger, Hollister, CA (US)

(73) Assignee: NEOVISTA, INC., Newark, CA (US)

(21) Appl. No.: 13/653,519

(22) Filed: Oct. 17, 2012

Related U.S. Application Data
(63) Continuation of application No. PCT/US2011/034109, filed on Apr. 27, 2011.
(60) Provisional application No. 61/328,517, filed on Apr. 27, 2010.

(51) Int. Cl. A61M 36/12; A61F 9/00

(52) U.S. Cl. 600/8

(57) ABSTRACT

A radiotherapy delivery device is provided comprising an elongated applicator having a proximal end and a distal end having a tip. The distal end is adapted to be introduced into a body including a treatment zone to be located adjacent a target tissue. A radiotherapy emitter is provided that is moveable between the proximal end of the applicator and the treatment zone of the applicator. The applicator includes a window to permit visual confirmation of the location of the radiation emitter and the treatment zone.

### FIELD OF THE DISCLOSURE

The present disclosure relates to a medical device and, more particularly, to a component of a device for delivering radiation therapy.

### BACKGROUND

Recently, novel methods and apparatus have been developed for radiation therapy in the treatment of macular degeneration. Such methods and apparatus may employ an applicator comprising a catheter or cannula for insertion into the eye for delivery and/or positioning of a radiotherapy source to the desired location. The applicator is positioned relative to the target tissue by viewing the cannula through the lens of the patient. Once the cannula is positioned, the radiotherapy source is delivered to the desired location where it remains for a period of time sufficient to deliver a therapeutic dose of radiation to the targeted tissue, after which the radiotherapy source is retracted from the catheter or cannula. See, e.g., de Juan Jr. et al. U.S. Pat. No. 6,875,165 and Larsen et al. U.S. Pat. No. 7,744,520, which are incorporated herein by reference.

To achieve suitably accurate dosing of the target tissue, and potentially avoid or limit excessive radiation of the non-target tissue, it is beneficial that the radiotherapy source, when advanced, be properly located, for example at the proper location within the treatment area of the applicator. The capability of achieving proper location can be checked, for example, with a test device, such as that shown in PCT/EP2004/012416, published as WO 2005/049140, in which the tip of an applicator forming a part of a radiation therapy delivery device (also referred to as a delivery cannula or catheter) is mounted in a holder having radiation detectors prior to use of the device on a patient. When a radiotherapy source is advanced in the applicator, the radiation detectors provide an indication that the radiotherapy source has reached the desired location for treatment. The radiotherapy source can then be withdrawn from the applicator, and the applicator removed from the test device and introduced into the body of the patient as part of its intended procedure with a reasonable confidence that the radiotherapy source, when introduced into the applicator, can be accurately advanced to the desired treatment site.

While testing outside of the body of the patient provides a reasonable assurance that the radiotherapy source will attain its desired location within the applicator cannula or catheter after the latter is positioned at the treatment site, the physician performing the treatment would prefer actual confirmation that the desired location of the radiotherapy source within the applicator has actually been attained during treatment, and not just during a pre-treatment testing of the applicator.

### SUMMARY OF THE DISCLOSURE

The present disclosure has a number of aspects which may be used in various combinations, and the disclosure of one or more specific embodiments is for the purposes of disclosure and description and not limitation. This summary only highlights a few of the aspects of this subject matter, and additional aspects are disclosed in the drawings and the more detailed description that follows.

By way of the present disclosure, an applicator comprising a cannula is provided that is suitable for performing intraocular brachytherapy and that permits confirmation by the physician as to whether radiotherapy source is properly positioned within the applicator for treatment when the cannula is located in the treatment position.

More particularly, a radiotherapy delivery device is provided comprising an elongated hollow applicator having a proximal end and a distal end having a tip. The distal end of the applicator is adapted to be introduced into a body and includes a treatment zone that is to be located adjacent a target tissue. A radiotherapy emitter is provided that is moveable within the applicator between the proximal end of the applicator and the treatment zone of the applicator. One of the applicator and the radiotherapy emitter is provided with a characteristic that permits the position of the radiotherapy emitter within the applicator to be determined. In one embodiment, the applicator includes a window to permit direct visual determination as to whether the radiation emitter is located in the treatment zone. Preferably, the window is transparent and also seals the interior of the applicator from the exterior.

In another aspect of the disclosure, the applicator comprises a stop for limiting the extent of distal movement of the radiotherapy emitter within the applicator.

In another aspect of the disclosure, the window may be located either proximally of the tip or may comprise the tip of the applicator.

In another aspect of the disclosure, the radiotherapy emitter comprises a canister having an elongated rod extending therefrom, the rod being visible in the window for visually assessing the location of the canister relative to the treatment zone.

In another aspect of the disclosure, the stop comprises a shoulder interior of the applicator, the shoulder defining an orifice through which the elongated rod passes into the tip of the applicator.

In another aspect of the disclosure, the canister abuts the shoulder when the radiotherapy emitter is properly located in the treatment zone.

In another aspect of the disclosure, at least one of the radiotherapy emitter and the applicator comprises a material that enhances the visual contrast between the two.

### DESCRIPTION OF THE DRAWINGS

FIG. 1 is a perspective view of a radiation delivery device comprising an applicator according to the present disclosure.

FIG. 2 is a cross sectional view of the distal tip portion of a first embodiment of an applicator according to the present disclosure.

FIG. 3 is a cross sectional view of a first alternate embodiment of the distal tip of an applicator according to the present disclosure.

FIG. 4 is a cross sectional view of a second alternative embodiment of an applicator tip according to the present disclosure.

FIG. 5 is a cross sectional view similar to FIG. 3, except that the radiation emitter is not properly located in the treatment position.

### DESCRIPTION

A more detailed description of a radiotherapy device in accordance with the present disclosure is set forth below. It should be understood that the description below of specific devices is intended to be exemplary, and not exhaustive of all possible variations. Thus, the scope of the disclosure is not intended to be limiting, and should be understood to encompass variations or embodiments that would occur to persons of ordinary skill.

A handheld device for performing intraocular brachytherapy is generally shown and described in U.S. patent application Ser. No. 12/477,228, filed Jun. 3, 2009, and published as US 2010/0030010, which is incorporated by reference. As disclosed in the referenced application, the radiation delivery device may have three basic components:
an elongated, flexible radiation source wire that has a radioactive isotope radiation emitter on the distal end thereof;
a handpiece for storing the radiation source wire and for moving the radiation emitter secured thereto between storage and treatment positions; and
a delivery cannula mounted to the distal end of the handpiece for introducing into the interior of the eye and for receiving the radiation emitter when the latter is advanced to the treatment position.

According to the present disclosure, an elongated delivery cannula or catheter is provided having a proximal end and a distal end. The distal end is adapted to be introduced into a body (e.g., the interior of the eye), and the cannula includes a treatment zone to be located adjacent a target tissue. A radiation emitter is movable within the cannula between the proximal end of the cannula and the treatment zone. In keeping with the disclosure, one of the cannula and the radiotherapy emitter is provided with a characteristic or structure that permits determination of the location of the radiotherapy emitter in the cannula when the cannula is in the treatment position. In one embodiment the cannula includes a viewing window or port on its distal end or in the treatment zone to permit visual determination as to whether the radiation emitter is in the desired location within the cannula. The window is preferably transparent and seals the distal end of the cannula so that no potentially-contaminating substances pass through the window between the exterior and interior of the cannula. When inserted into an eye, the cannula may be viewed through the lens of the eye using a medium power monocular. The cannula preferably includes an interfering structure such as a stop to positively locate the radiation emitter and to limit the extent to which it can be advanced distally within the applicator. In addition, the cannula and emitter may be of a contrasting color or finish to enhance visual confirmation of proper location. For example, at least one of the radiation emitter and the applicator may be coated with or comprise a material that enhances the visual contrast between the two.

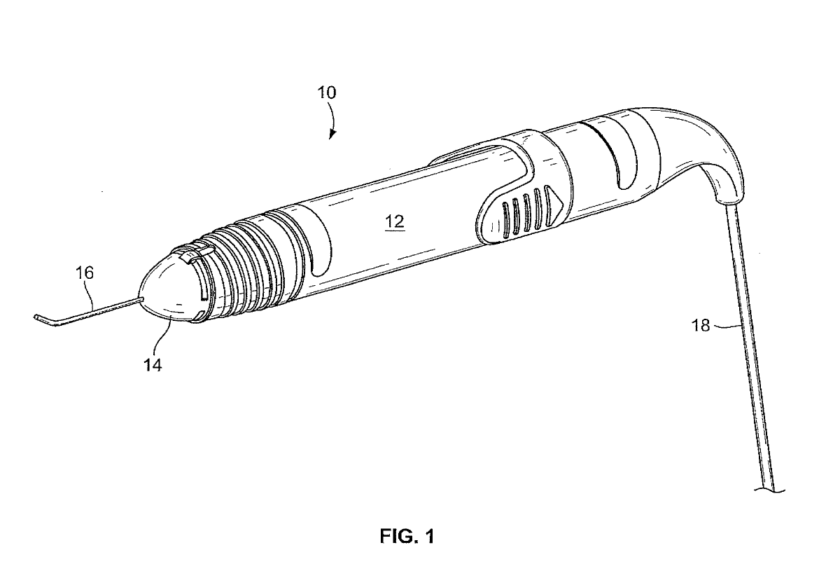

Turning to FIG. 1, a radiation delivery device, generally designated 10, is shown that may advantageously incorporate the visual confirmation window disclosed herein. The device 10 includes a handpiece or delivery module 12 with a removable applicator tip 14 (preferably disposable) secured to its distal end. The applicator tip 14 includes a cannula 16 sealed at its distal end and having a hollow interior. The handpiece 12 encloses an assembly that mounts the radiation source wire or “RSW” (not shown) to facilitate its movement between a retracted/storage position, in which the RSW resides completely within the handpeice, and an extended/treatment position in which the radiation emitter on the RSW is located at a predetermined treatment position within the cannula 16. In the illustrated device, the RSW is moved between the retracted/storage position and the extended/treatment position by means of a remote actuator (not shown) operatively connected to the handpiece 12 by an umbilicus 18. A more detailed description of the delivery system may be found in the above-referenced published application US 2010/0030010.

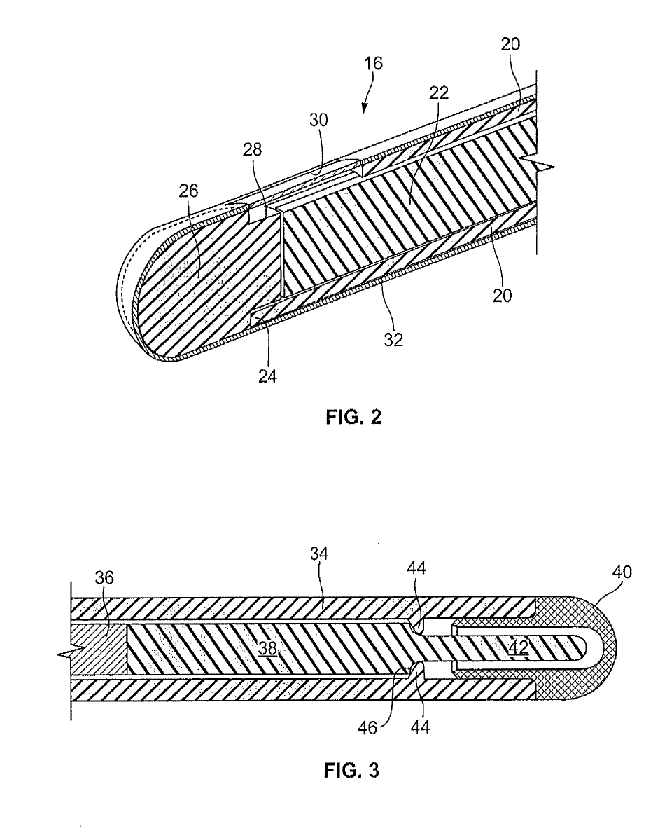

Turning to FIG. 2, a first embodiment of a cannula having a visual confirmation window in accordance with the present disclosure is shown. The cannula 16 preferably comprises a biocompatible material, and is preferably a relatively rigid tubular member 20 made of a material such as a stainless steel or rigid plastic. The tubular member 20 has a lumen sized to receive on the interior thereof a radiotherapy emitter 22 (which may of any suitable radioactive emitting material or device, such as a beta or gamma emitter, or an x-ray emitter). The radioactive material is preferably contained within a canister made of, e.g., stainless steel that comprises the distal end of the RSW. The proximal end of the tubular member 20 may be configured such that the tubular member is removably secured to a handpiece 12 as described above. The distal end 24 of the tubular member 20 is closed by a tip member 26 having a proximally-extending portion 28 that is received in the distal interior of the tubular member 20 and is secured thereto by, e.g., welding or an adhesive. As illustrated, the portion of the tip 26 external to the tubular member 20 has a semi-spherical, atraumatic configuration, although this is not required. In addition, the outside diameter of the tip member 26 is the same as the outside diameter of the tubular member so as to provide a smooth transition between the two.

In the embodiment of FIG. 2, the portion 28 of the tip 26 received on the interior of the tubular member acts as a stop for the radiotherapy emitter 22 when the emitter is located in the treatment position. That is, the radiotherapy emitter 22 abuts the proximal-facing face of the portion 28 of the tip 26 on the interior of the tubular member 20 when advanced to the treatment position (as shown in FIG. 2).

By way of the present disclosure, the tubular member 20 may also be provided with a viewing port or window 30 that permits direct visual observation of the interior of the tubular member to ascertain when the radiotherapy source is located at the treatment position of the cannula. As illustrated, for example, in FIG. 2, the window is located adjacent and proximal to the stop for the radiation emitter 22 when the latter is in the treatment position. The window 30 is preferably sealed, but still transparent or translucent, so that no fluid passes through the window 30 into the interior of tubular member 20.

The window 30 may be provided or formed in any suitable manner or with any suitable size or shape to allow visual detection of the presence or absence of the radiotherapy source in the treatment zone. As shown in FIG. 2, the window 30 is formed by partially circumferentially removing a portion of the wall of the tubular member 20 to expose the interface of the distal end of the tubular member 20 with the proximal portion 28 of the tip member 26 that provides the abutment surface for the radiation emitter 22 when in the treatment position. The window 30 is located radially on the tube wall so that it faces the lens of the eye, and thus is visible to the physician viewing the cannula through the lens of the patient; i.e., the window 30 is on the “top side” of the tubular member, as seen in FIG. 2. The window 30 may be created by cutting, grinding or molding, although other material removal techniques may be used. Further, while the window 30 is described as being located at the junction of the tip 26 member and the distal end of the tubular member 20, the window 30 may be located anywhere along the length of the cannula to provide visual confirmation (direct or indirect) of correct positioning of the radiotherapy source within the treatment zone, e.g., the window may be located where there is a stop formed for engagement by the RSW when the radiation emitter is in the treatment position.

The window 30 may be covered or otherwise sealed with any suitable material that both forms a fluid-tight seal and is sufficiently transparent so that the radiation emitter can be seen through the window when located in the treatment position. In one embodiment, a covering or cap 32 may be formed separately of a medical grade, UV-cured coating/adhesive (such as UV 18 MED, manufactured by Master Bond Inc., of Hackensack, N.J.). To form the cap 32, a mandrel having substantially the same dimension and shape as the distal portion of the cannula 16 and the tip may be dipped in the coating material, the coating cured and the cured coating that comprises the cap 32 removed from the mandrel. An adhesive is applied to the cannula proximally of the window, and the cap is applied to the cannula and tip. The cap 32 may be relatively thin, such as a thickness on the order of 0.001 inches, so as to provide a low profile at the transition point along the length of the cannula 16 between the outside diameter of the cannula 116 and the outside diameter of the cap 32. Alternatively, a thin-wall, PET, non-shrinkable tubing (available from Advanced Polymers Incorporated, 29 Northwestern Drive, Salem, N.H.) can be used to seal the window.

It may be also be desirable to enhance the visual contrast at the window between the radiation emitter and the stop. For example, the exposed surface of the stop in the interior of the tubular member could be coated with a fluorescent material, which is partially obscured by the radiotherapy emitter 22 when the latter abuts the stop. Similarly, the cannula and radiotherapy emitter could be provided with finishes or made of materials having differing reflective characteristics to provide a visual contrast. Other techniques may also be employed to enhance visual confirmation, e.g., a magnifying lens located within the window to enhance user visual identification of the presence of a radiotherapy source.

In an alternative embodiment, instead of there being a window in the sidewall of the cannula, the cannula may be provided with a clear or transparent tip, through which the position of the canister containing the radiation emitter can be visually determined. The clear tip may include a hollow interior portion that receives the source can when the RSW is in the extended/treatment position. More preferably, the source can for the radiation emitter may include an extension, such as an elongated rod or pin, on its distal end which is received within the hollow interior portion of the tip when the RSW is properly located in the extended/treatment position. Thus, the relative length of the elongated rod that is visible through the tip provides a visual indication of whether the source can for the radiation emitter is properly located in the treatment zone.

Turning to FIG. 3, there is seen the distal end of a cannula 34 and an RSW 36 having a radiation emitter housed in a source can 38. The distal end of the cannula 34 is provided with a clear tip 40, preferably made of a plastic material, such as acrylic, polycarbonate, or any other clear, biocompatible material. The tip 40 is secured to the cannula by, e.g., any suitable adhesive. The tip 40 has a hollow interior portion into which a portion of the source can 38 is received and is visible when the radiation emitter is advanced to the treatment position. As illustrated, the source can 38 preferably has an elongated rod or pin 42 secured to its distal lid that becomes visible through the plastic tip when the radiation emitter is deployed. If the radiation emitter is successfully deployed, the portion of the pin 42 visible through the tip 40 is greater than that portion which is visible if the radiation emitter is undeployed or of there has been a failed deployment, as seen by comparing FIG. 3 (successfully deployed) to FIG. 5 (failed/incomplete deployment).

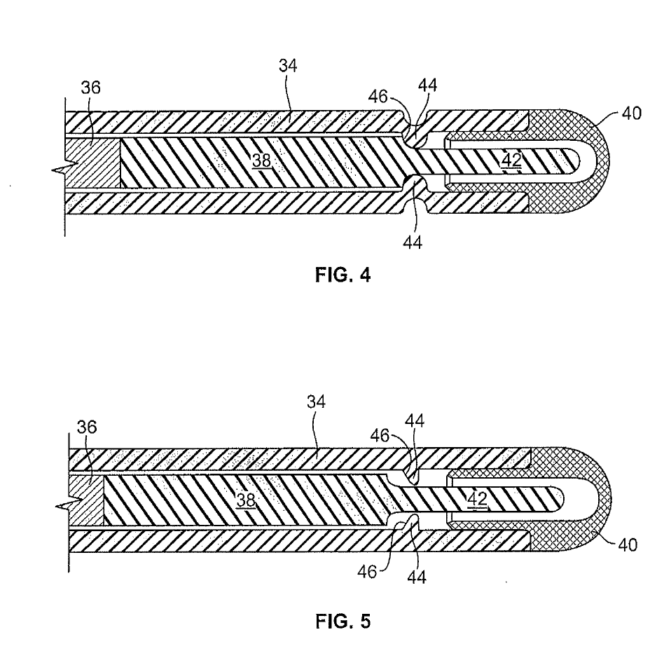

In order to reduce the likelihood of the dislodgement of the tip 40 by the advancement of the RSW 36, it is preferable that neither the source can 38, nor its associated pin 42, contact the inner surface of the tip 40 during advancement of the radiation emitter to the treatment position. To this end, the cannula 34 may be provided with an internal stop or shoulder 44 that is engaged by the distal lid of the source can 38 when the radiation emitter is fully deployed, such that the distal end of the pin 42 is still spaced from the interior surface of the tip 40. As seen in FIGS. 3 and 5, the stop 44 may take the form of an annulus or ring that is either secured to or formed integrally with the inner surface of the cannula. Alternatively, the integral stop 44 may be provided by swaging or crimping the tubing forming the cannula 34, so that it is radially indented to provide a narrowed waist, as seen in FIG. 4. In either case, the proximal portion of the stop 44 preferably presents a tapered or funnel-like surface 46 so that if it is contacted by the end of the pin 42 during the advancement of the RSW to the deployed position, the pin is smoothly guided through the opening in the stop 44 into the hollow portion of the clear tip 40. Also, all the potential points of contact on the source can 38 with the stop (such as the distal end of the pin 42, the attachment point of the pin 42 to the source can 38, and edges of the distal lid of the source can 38) may also be rounded or radiused to provide for smooth movement of the RSW toward the deployed position.

Thus, a radiotherapy cannula with a visual confirmation window has been provided. While the radiotherapy cannula has been described in terms of certain preferred embodiments, it is not limited to these specific embodiments, and a variety of changes may be made as apparent to a person of ordinary skill in the art without departing from the concepts disclosed herein. For example, while the cannula has been described as having a window to permit visual confirmation of the location of the radiotherapy emitter, the emitter may have other signal generating or reflective characteristics that permit its location at the treatment position of the cannula to be determined.

1. A radiotherapy delivery device comprising:
an elongated hollow applicator having a proximal end and a distal end having a tip, the distal end being adapted to be introduced into a body and including a treatment zone to be located adjacent a target tissue;
a radiotherapy emitter receivable within the applicator and movable between the proximal end of the applicator and the treatment zone of the applicator;
the applicator further comprising a window to permit visual confirmation as to whether the radiation emitter is located in the treatment zone simultaneously with the applicator being positioned in the treatment zone.

2. (canceled)

3. The radiotherapy device of claim 1 wherein the window is transparent and provides a seal between the interior and exterior of the applicator.

4. The radiotherapy delivery device of claim 3 wherein the applicator further comprises a stop for limiting the extent of distal movement of the radiotherapy emitter.

5. The radiotherapy device of claim 4 in which the window comprises the tip of the applicator.

6. The radiotherapy device of claim 4 wherein the window is located proximally of the tip.

7. The radiotherapy device of claim 4 wherein the radiotherapy emitter comprises a canister with an elongated rod extending therefrom, the rod being visible in the window for visually assessing the location of the canister relative to the treatment zone.

8. The radiotherapy device of claim 7 wherein the stop comprises a shoulder interior of the applicator, the shoulder defining an orifice through which the elongated rod passes into the tip of the applicator.

9. The radiotherapy device of claim 8 wherein the canister abuts the shoulder when the radiotherapy emitter is properly located in the treatment zone.

10. The radiotherapy device of claim 9 wherein the tip includes a hollow interior defining a passageway into which the elongated rod is received, the passageway having a distal end surface, and the canister abutting the shoulder on the interior of the applicator before the elongated rod contacts the distal end surface of the passageway in the tip.

11. The radiotherapy device of claim 1 wherein at least one of the radiotherapy emitter and the applicator comprises a material that enhances the detectable contrast between the two.
